(19) United States
(12) Patent Application Publication
(10) Pub. No.: US 2006/0074348 A1
(43) Pub. Date: Apr. 6, 2006

(54) Biologic Fluid Sampling Apparatus

(75) Inventors: Jeffrey Lee Spray, Erie, CO (US); Todd Curtis Green, Lakewood, CO (US); Larry Joe Dumont, Arvada, CO (US)

Correspondence Address: GAMBRO, INC;PATENT DEPARTMENT, 10810 W COLLINS AVE, LAKEWOOD, CO, 80215, US

(73) Assignee: GAMBRO, INC., Lakewood, CO (US)

(21) Appl. No.: 10/711,707

(22) Filed: Sep. 30, 2004

(51) Int. Cl. A61B 5/00; A61B 19/00

(52) U.S. Cl. 600/576; 604/408

(57) ABSTRACT

A blood sampling device having a valve between a chamber for receiving a limited quantity of fluid and a needle for penetrating a sample bottle. The valve comprises a rigid connector with a compressible tube extending along a beam and an arm extending across tube and the beam such that the tube can be compressed between the beam and the arm. A bottle adapter connected to the valve has an elongated or ovoid base. A needle is enclosed within the bottle adapter. Within the bottle adapter are longitudinally extending fins. A removable needle cap over the needle is held in place by a lid. Another bottle adapter has a wall deformable from a first configuration into a second configuration. The bottle adaptor may be removable and may have a slot such that the needle and the needle cap can be inserted into the bottle adaptor through the slot.

### BACKGROUND OF INVENTION

This invention relates to collection of biological fluids, and in particular to apparatus for the collection of one or more samples of a biological fluid for analysis. More particularly the invention relates to collection of blood from donors and extraction from the collected blood of one or more samples.

During a blood donation, samples of blood for analysis should be taken in such a way that contamination of the collected blood cannot occur through the process of sample collection. It is standard practice to take samples after a desired amount of blood has been collected and after the line to the collection bags has been sealed. Samples can then be taken by using sampling ports, by piercing the blood-line with a sampling needle, or by dripping blood into open collection vials at any point between the IV needle (which is still in the donor's vein) and the point at which the line has been sealed.

Bulbs or bag containers for collecting blood samples are shown, for example, in U.S. Pat. No. 5,496,301 to Hlavinka et al., as well as in PCT/US99/10152 (published Nov. 18, 1999 as WO99/58094) and U.S. application Ser. No. 10/428,513 (published Jan. 15, 2004 as US2004-0009542 A1), all now commonly owned by Gambro, Inc.

Another example of known blood sampling methods in blood collection procedures is provided in the disclosure of U.S. Pat. No. 4,786,286 by Baxter International Inc. This publication and WO 94/12093, also by Baxter, disclose the use of in-line containers of various designs for blood sample collection after blood collection has been completed. In WO 90/12606, Baxter discloses the use of pre-slit septum ports for blood sampling during infusion techniques. Other in-line sampling ports are disclosed by Spacelabs Inc in European patent publication No. 0 376 168.

U.S. Pat. No. 3,654,924 to Wilson et al. teaches the use of a flow-through sample pouch formed around a portion of the blood line that includes a frangible connection so that, if a sample is required, the connection can be broken and blood allowed to flow into the sample pouch. U.S. Pat. No. 5,167,656 to Lynn also teaches the use of a flow-through sample-pouch but omits the use of the frangible connection in the blood line. Lynn's pouch fills with blood as blood flows to the bag-set and, after a unit of blood has been collected in the set, the line up-stream and down-stream from the pouch is sealed and the IV needle is withdrawn. Samples of blood may then be taken from the pouch. The use of such flow-through pouches has the advantage that the procedure is shorter for the donor because samples can be taken from the pouch after the needle is removed from the donor's arm.

U.S. Pat. No. 4,056,101 to Geissler discloses the use of an in-line trap that is set to collect the first few milliliters (ml) of blood drawn from a donor. It is manually operable by pulling on the blood line to cause the remainder of blood flowing from the donor to pass to the bag-set.

In Japanese patent application No. 09028265 (Publication No. 10211274), Terumo Corp, teaches the use of a first in-line frangible seal in the blood line between the IV needle and the blood bag and a second frangible seal in a branch sample line connected to the blood line upstream of the first connector. Each seal blocks its respective line until it is broken by external manipulation of the line. After the IV needle is inserted in the donor, the second seal is broken to allow blood to flow into the sample line for removal via a sample port connected to the sample line. The first seal is then broken to permit flow of blood to the collection bags.

U.S. Pat. No. 6,626,884 to Dillon involves the use of a multi-port/multi-position valve in the blood line from the needle to the collection bag that can be operated to first connect the needle line to a branch line at the start of the blood collection procedure and then connect the needle line to the bag line for blood collection. The sample container may be rigid or semi-rigid, and may have a filtered vent with automatic shut-off for allowing air to escape. Such vents are well known in the art.

In addition to apparatus disclosed in patents, various devices for taking samples of blood or other biologic fluids are commercially available. Such apparatus include the SampLok™ Sampling Kit from ITL Corporation of Australia, the Gambro BCT Sample Bag System, the CharterMed Bag System, and the MacoPharma Bag System.

Prior art devices can be further improved, however. For example, it is desirable to dispense accurate quantities of sampled blood or other biologic fluid from a temporary reservoir into a sample bottle. In addition, certain features of prior art devices make them more difficult to store or ship or to adapt to different configurations of sample bottles.

### SUMMARY OF INVENTION

The apparatus of the present invention provides a blood or biologic fluid sampling device having an integrated valve interposed between a chamber for temporarily receiving a limited quantity of fluid and a needle for penetrating a fluid sample bottle, whereby the quantity of fluid dispensed into the sample bottle can be more accurately controlled. Another aspect of the invention is that the apparatus can be pre-assembled and can be more easily stored or used in blood processing devices such as incubators by reason of the shape of a bottle cap adapter of elongated or ovoid shape. Yet another feature of the invention is a biologic fluid sampling device with a removable bottle cap adapter so that the residue of the sampling device can be transported and stored separately from the bottle cap adapter. Moreover, a particular bottle cap adapter can be selected from a set of adapters to conform to an available type of sample bottle.

One aspect of the present invention, therefore, is to provide a biologic fluid sampling device comprising a receptacle having an interior chamber for receiving a biologic fluid, a fluid access port, a fluid egress port, and an air vent, a needle in fluid communication with the chamber through the fluid egress port, and a valve interposed between the chamber and the needle whereby flow of biologic fluid from the chamber through the needle may be controlled. The sampling device may further comprise a rigid base or connector supporting the valve. The base or connector may have a compressible tube extending along a central beam from the fluid egress port to the needle. The valve may have an arm pivotally connected to the rigid connector and extending across tube and the central beam such that the tube can be compressed between the beam and the arm. A ridge may extend along the arm, to selectively press against the tube.

The sampling device may also comprise a bottle adapter connected to the valve and the needle. In one embodiment, the bottle adapter has an elongated base and a wall connected to the base, the wall extending further than the needle such that the needle is effectively enclosed within the bottle adapter. Within the bottle adapter a plurality of longitudinally extending fins form a guide to direct the cap of a sample bottle onto the needle. A removable needle cap over the needle protects a user from inadvertent injury. The needle cap may be held in place by a lid. The needle cap also has a grip, preferably extending through the lid that allows the needle cap to be removed without a user putting any part of the user's hand into the bottle cap adapter.

In another embodiment, the bottle adapter has a base and a wall connected to the base and extending along the needle such that the needle is enclosed within the bottle adaptor, and the wall is deformable from a first configuration, for example ovoid or elliptical, into a second configuration, for example, circular. The bottle adaptor may have a plurality of expandable folds, the folds being symmetrically spaced around the wall.

The bottle adaptor may also have a lid pivotally connected to the wall by a hinge and a clasp connected to the wall for securing the lid in a closed position. A frangible valve may be used in the access port of the receptacle.

In a further embodiment, the sampling device may have a coupling at a proximal end of the needle, a needle cap removably fitted over the needle and releasably connected to the coupling, and a bottle adaptor may be configured to removably connect to the coupling. The bottle adaptor may have a centrally located bore, the coupling being adapted to fit within the bore, and a slot extending lengthwise from the bore such that the needle and the needle cap can be inserted into the bottle adaptor through the slot.

Further advantages and features of the present invention will be apparent from the following detailed description together with the accompanying drawings.

### BRIEF DESCRIPTION OF DRAWINGS

FIG. 1 is a plan view of a biologic fluid sampling apparatus according to the present invention.

FIG. 2 is a plan view of a fluid sampler from the apparatus of FIG. 1.

FIG. 3 is a top perspective view of a reservoir and pinch clamp according to the present invention.

FIG. 4 is a bottom perspective view of the reservoir and pinch clamp of FIG. 3.

FIG. 5 is a perspective view of a first embodiment of a bottle cap adapter.

FIG. 6 is a bottom perspective view of the first bottle cap adapter of FIG. 5.

FIG. 7A is a through section of the bottle cap adapter of FIG. 5, taken along line 7-7.

FIG. 7B is a through section similar to FIG. 7A, also showing a needle cap and closed lid, the needle cap having a grip extending through a slot in the lid.

FIG. 8 is a perspective view of a needle cap, used in connection with the bottle cap adapter of FIG. 5.

FIG. 9 is a through section of the needle cap of FIG. 8, taken along line 9-9.

FIG. 10 is a perspective view of a second embodiment of a bottle cap adapter.

FIG. 11 is a bottom perspective view of the second bottle cap adapter of FIG. 10.

FIG. 12 is a through section of the second bottle cap adapter of FIG. 10, taken along line 12-12.

FIG. 13 is a perspective view of a second needle cap, used in connection with the second bottle cap adapter of FIG. 10.

FIG. 14 is a bottom perspective view of the second needle cap of FIG. 13.

FIG. 15 is a perspective view of a third embodiment of a bottle cap adapter.

FIG. 16 is a top plan view of the third bottle cap adapter of FIG. 15.

FIG. 17 is a front plan view of the third needle bottle cap adapter of FIG. 15.

FIG. 18 is a perspective view of a reservoir, needle and needle cap, for use with the third bottle cap adapter of FIG. 15.

FIG. 19 is a partial plan view of the reservoir, needle and needle cap of FIG. 18, with needle cap partially removed, and through section of the third bottle cap adapter of FIG. 15, taken along line 19-19.

### DETAILED DESCRIPTION

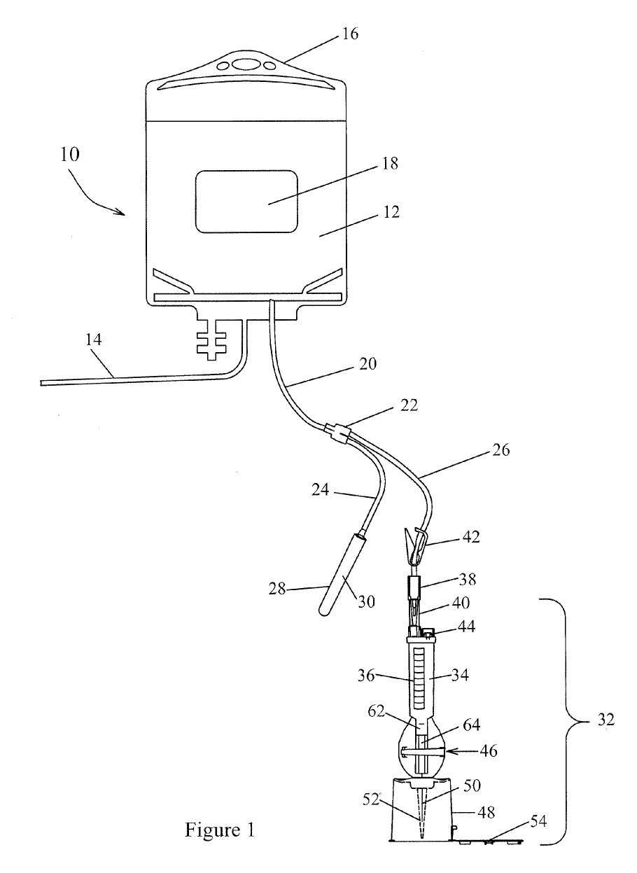

Referring now to the drawings, FIG. 1 illustrates a bacterial sampling apparatus 10. The apparatus 10 comprises a component bag or container 12 for receiving a biological fluid such as blood. The component bag 12 may have an access line 14 that allows the blood or other fluid to be conducted into and out of the bag 12 from a fluid source such as a donor, patient, blood-processing machine, apheresis machine or some other apparatus (not shown). Conventional features of component bags include support tabs 16 for hanging the bag and labels 18.

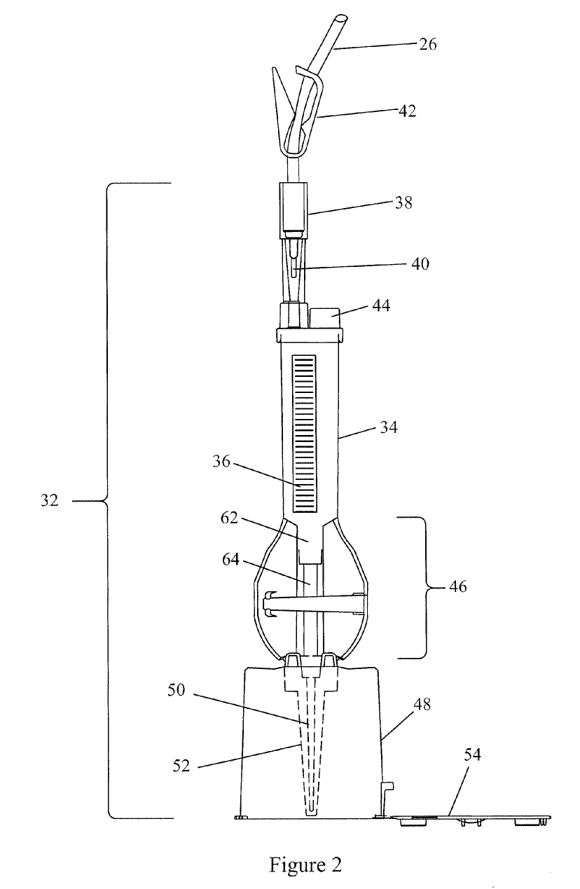

A sampling tube 20 connects the bag 12 to a Y-connector 22. The Y-connector 22 divides the sampling tube 20 into a first tube 24 and a second tube 26. The first tube 24 connects to a platelet product sampler 28 comprising a vial 30 for receiving a sample of collected biologic product, for example, platelets. The second tube 26 connects the component bag 12 to a fluid sampler 32 by way of the Y-adapter 22. As shown both in FIG. 1 and also in FIG. 2 in greater detail, the fluid sampler 32 of the present invention comprises a reservoir 34, preferably formed of transparent biocompatible plastic and having thereon a gauge 36 whereby a level of fluid in the reservoir can be measured by reference to the gauge 36. A fluid access port 38 containing a frangible pin 40 connects the second tube 26 to the reservoir 34. A pinch clamp 42 placed on the second tube 26 controls flow of fluid into the reservoir 34. The pinch clamp 42 is of conventional design and need not be described fully here. An air vent 44 allows air to enter and escape from the reservoir 34, as more fully described hereafter.

A flow control valve 46, also described more fully hereafter, connects the reservoir 34 to a bottle adapter 48 and needle 50 (shown in FIG. 2). A removable needle cap 52 covers the needle 50. A lid 54 closes over the needle cap 48 and holds the needle cap 52 over the needle 50 prior to use.

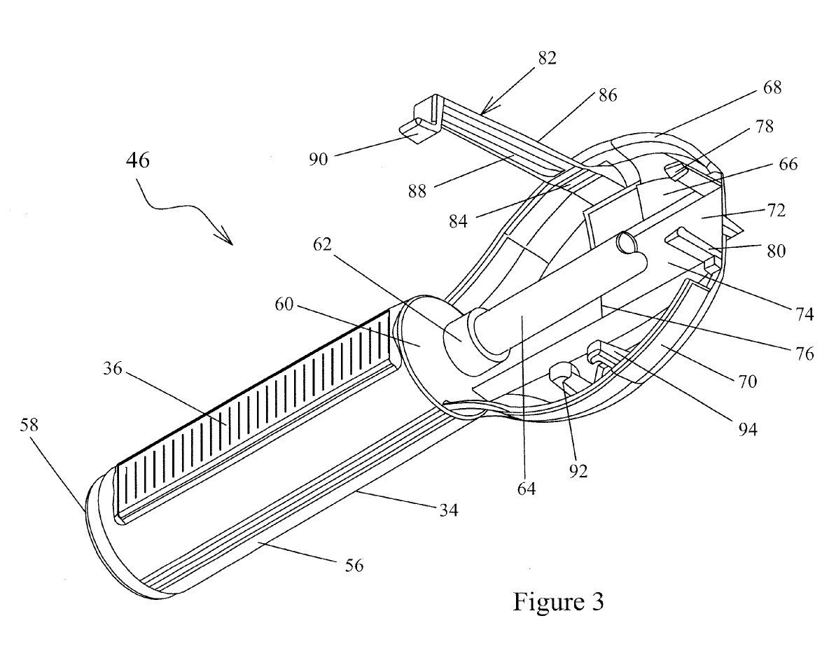

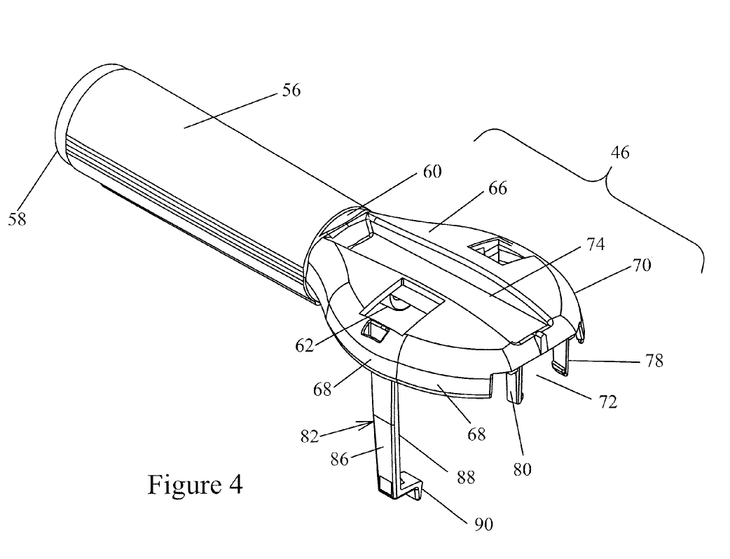

The assembly of the biologically compatible reservoir 34 and flow control valve 46 can be seen more clearly in FIG. 3 and FIG. 4. Preferably, the reservoir 34 comprises a cylindrical container or chamber 56 having a proximal end 58 with a fluid access port (not shown in FIG. 3 or FIG. 4) and a distal end 60. At the distal end 60, a centrally located fluid egress port 62 receives a flexible tube segment 64, which communicates with the needle 50 (see FIG. 2, not shown in FIG. 3 or FIG. 4). The flow control valve 46 also connects to the distal end 60 of the cylindrical container 56 and comprises a rigid, generally elliptical base 66 with left and right walls 68, 70, which curve along edges of the base 66 from the distal end 60 of the container 56 to an opening 72 opposite the female connector 62. A beam 74 extends from one side of the female connector 62 along the base 66 to the opening 72 and provides a supporting surface for the flexible tube segment 64. Preferably the beam 74 has a transverse ridge 76 midway along the beam 74 and at right angles to the flexible tube segment 64. First and second latches 78, 80 bracket the beam 74 at each side of the opening 72 and adjacent ends of the left wall 68 and the right wall 70, respectively. A clamp arm 82 has a fixed end that pivotally connects to the base 66 at a hinge 84 adjacent to and midway along the left wall 68. The clamp arm 82 comprises a relatively rigid bar 86 having a longitudinal ridge 88 extending transversely to the tube segment 64 such that the longitudinal ridge 88 passes over the transverse ridge 76 on the beam 74. At the other end of the bar 86 from the hinge 84, an offset tab 90 engages a bracket comprised of two opposed latches 92, 94 adjacent to and midway along the right wall 70 of the flow control valve 46. In use, the resilience of the flexible tube segment 64 raises the bar 86 away from the beam 74 and allows fluid to flow through the tube segment 64 and the needle 50 into a medical sample bottle (not shown). Finger pressure on the base 66 and the clamp arm 82 will force the longitudinal ridge 88 and the transverse ridge 76 together, pinching the tube segment 64 and interrupting fluid flow.

The vent 44 on the reservoir 34 (see FIG. 1 and FIG. 2) allows air to pass into and out of the reservoir as biologic fluid, such as blood, flows into and out of the reservoir. A filter, such as a 0.2 micron filter available from Borla, Inc. or Industrie Borla S.p.A., Moncalieri, Italy, prevents bacterial contamination of the fluid in the reservoir.

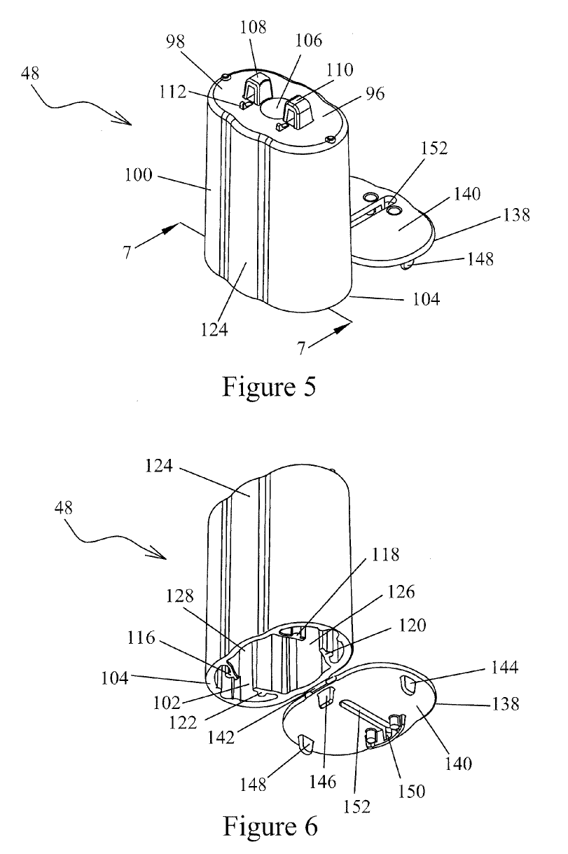

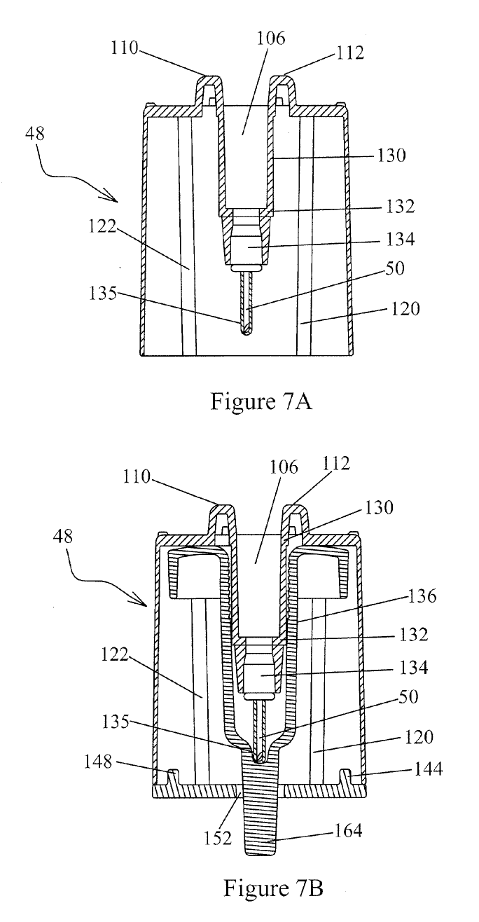

The presently preferred configuration of the bottle adapter 48 is illustrated in FIG. 5, FIG. 6, FIG. 7A and FIG. 7B. The bottle adapter 48 comprises a generally ovoid or elongated base 96 forming a proximal end 98 of the bottle adapter 48. A wall 100 extends from the base 98, forming a cavity 102. A distal end 104 of the bottle adapter 48 is open to receive the mouth of a medical sample bottle (not shown). As is well known, medical sample bottles often have a piercable septum that the needle 50 can penetrate for sterile transfer of fluid into the bottle. The base 98 has a central through bore 106 through which a needle can be inserted, as more fully explained below. On opposing sides of the through bore 106 are a pair of brackets 108, 110 that connect to the latches 78, 80 of the flow control valve 46. Each bracket 108, 110 has a stop 112, 114 associated with it.

Inside the bottle adapter 48 are four inwardly extending fins 116, 118, 120 and 122 extending along the length of the wall 100. Between a pair of fins 116, 118 on one side of the wall 100, the wall 100 bulges outwardly forming an arched wall segment 124. Similarly, between a pair of fins 120, 122 on an opposite side of the wall 100, the wall 100 also bulges outwardly forming an arched wall segment 126. Together the four fins 116, 118, 120, 122 and the arched wall segments 124, 126 form a cylindrical area 128 within the bottle adaptor 48 and centered around the needle 50. The cylindrical area 128 is sized to snuggly receive a bottle cap of a sample bottle and to guide the cap with its piercable septum onto the centrally located needle 50.

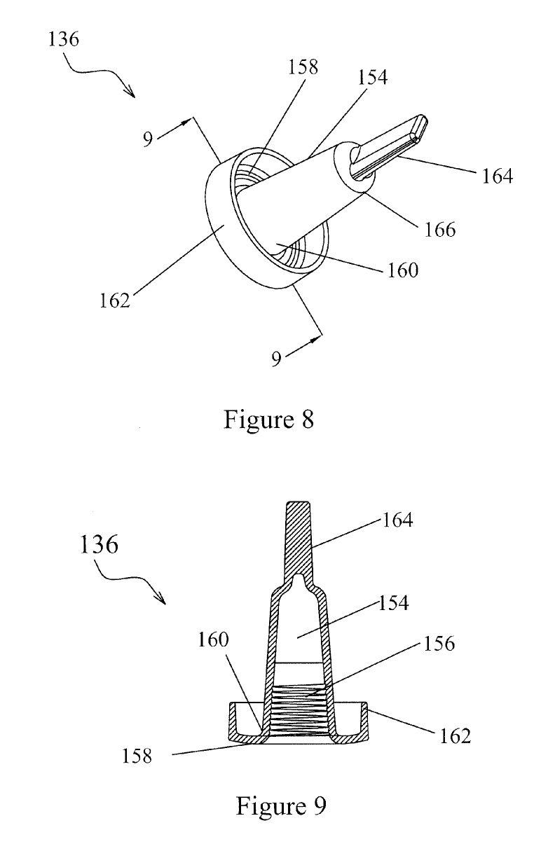

Also within the bottle adapter 48, a cylindrical receptacle 130 extends inwardly from the through bore 106, providing a secure connection for the flexible tube segment 64. At a distal end 132 of the receptacle 130, a socket or needle mount 134 supports the needle 50. The needle 50 is sized to terminate within the bottle adapter 48, reducing the chance of inadvertent injury to a user of the device. In addition to a needle cap, an elastomeric sleeve 135 may also cover the needle 50, as is known in the art. When the needle is pushed into a sample bottle, the needle also penetrates through the sleeve 135, which is pushed back along the shaft of the needle. As the needle is withdrawn, the sleeve springs back into its original shape, covering the needle. For further protection, the needle 50 is covered by a needle cap 136, illustrated in FIG. 8 and FIG. 9, which is held in place within the bottle adapter 48 by a lid 138 on the bottle adapter 48. The lid 138 comprises a generally flat plate 140 configured to conform to the distal end 104 of the bottle adapter 48 and attached to the bottle adapter 48 by a hinge 142. Tabs 144, 146, 148 on the plate 140 contact inner surfaces of the wall 100 when the lid is closed. A latch 150, located opposite the hinge 142 on the plate 140 frictionally closes the lid 138. A removable or breakable seal (not shown) may also be provided to assure that the lid is not opened until the apparatus is to be used. A slot 152 in the lid 138 extends from the latch 150 across the lid 138 towards the hinge 142 and accommodates the needle cap 136, as explained below.

The needle cap 136 (FIG. 8 and FIG. 9) comprises a chamber 154 that fits over the needle 50. A tortuous path engagement 156 fits over the cylindrical receptacle 130, inhibiting bacterial or other contamination of the needle 50. A flange 158 at a proximal end 160 of the chamber 154 with a circumferential lip 162 also supports the needle cap 136 by engaging the flanges 116, 118, 120, 122 in the bottle adapter 48. A finger grip 164 at a distal end 166 of the chamber 154 fits through the slot 152 in the lid 138 when the lid is closed. The slot 152 is long enough so that the lid may be opened with the needle cap 136 in place within the bottle adapter. Once the lid has been opened, a user may grasp the finger grip 164 and pull the needle cap 136 out of the bottle adapter 48, thereby exposing the needle 50. The needle 50 is still surrounded by the walls 100 of the bottle adapter 48 even with the needle cap 136 removed.

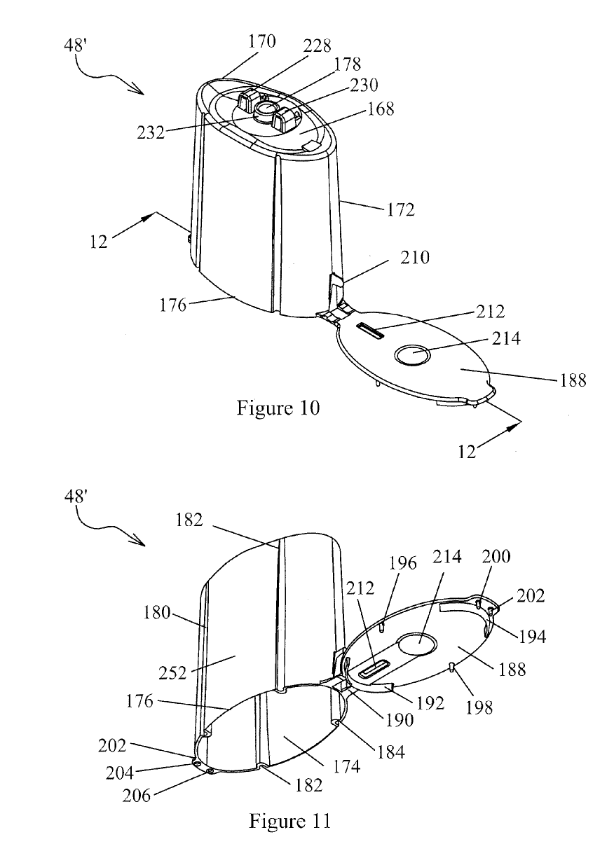

Another embodiment of a bottle adapter 48′ is illustrated in FIGS. 10 through 14. As explained above, the bottle adapter 48′ also comprises a generally ovoid or elongated base 168 forming a proximal end 170 of the bottle adapter 48′. A wall 172 extends from the base 168, forming a cavity 174. A distal end 176 of the bottle adapter 48′ is open to receive the mouth of a medical sample bottle (not shown). As mentioned above, medical sample bottles often have a piercable septum that the needle 50 can penetrate for sterile transfer of fluid into the bottle. The base 168 has a central through bore 178 through which a needle can be inserted. In this embodiment, however, the bottle adapter 48′ is configured to receive wide-mouth sample bottles, while still ordinarily maintaining an ovoid or elliptical configuration that is more easily manipulable and which is easier to store and ship in a platelet incubator. For example, in certain incubators commonly found in blood processing laboratories, the shelf spacing in the agitator of the incubator is constrained. The elongated shape of the bottle adapter fits more easily into such devices. Expandable folds 180, 182, 184, 186 symmetrically spaced in the wall 172 allow the wall to expand into a round or cylindrical configuration near the distal end 176 of the bottle adapter 48′. The wall 172 is preferably longer than the first embodiment described above to accommodate this distortion at the distal end 176 while constrained in an ovoid shape at the proximal end 170 by the base 168.

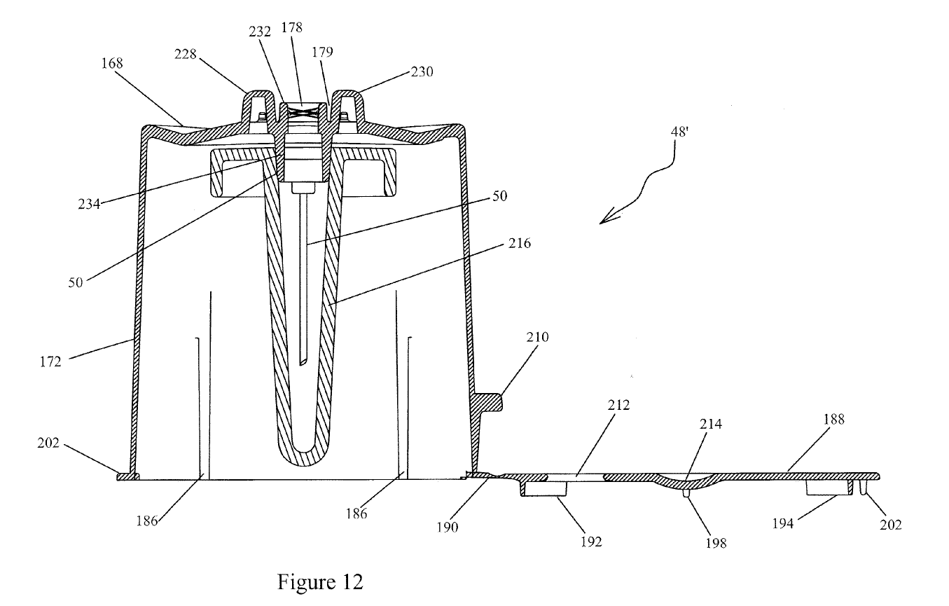

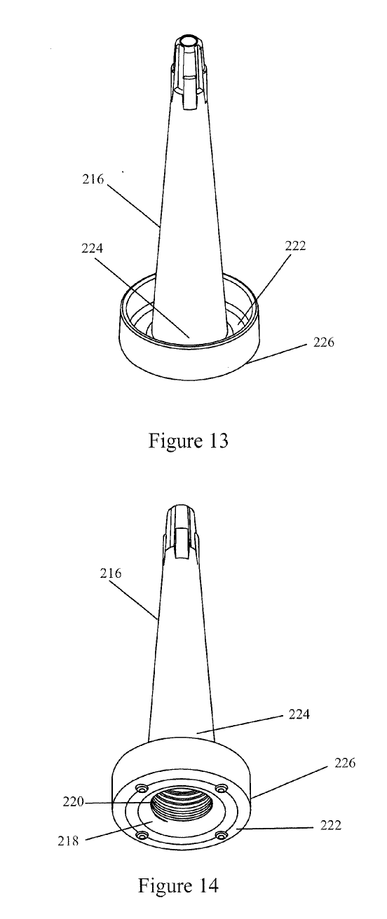

An ovoid lid 188 attaches to the distal end 176 of the wall 172 at a hinge 190 such that the lid preferably closes along the long axis of the distal end 176 when the distal end is in an ovoid or elliptical configuration. A first curved lip 192 adjacent the hinge 190 and a second curved lip 194 opposite the first curved lip hold the distal end 176 of the wall 172 in its ovoid shape when the lid 188 is closed by extending the long axis of the distal end 176. Pegs or tabs 196, 198 may be provided on the lid 188 to engage the wall 172 along the short axis of the distal end 176 when the distal end is in the ovoid or elliptical configuration. Additional pins or tabs 200, 202 near the second curved lip 194 may also engage sockets 204, 206 in an abutment 208 on the wall 172 opposite the hinge 190 to hold the lid 188 closed. As above, a removable or breakable seal (not shown) may also be provided to assure that the lid is not opened until the apparatus is to be used. The lid 188 folds back against the wall 172 where a hook or latch 210 secures the lid 188 by engaging a slot or catch 212 in the lid. A protrusion 214 in the center of the lid 188 extends slightly into the cavity 174 of the bottle adapter 48′ when the lid 188 is closed. With the lid in the closed position, the protrusion 214 presses against an elongated needle cap 216, shown in FIG. 12, FIG. 13 and FIG. 14.

The elongated needle cap 216, like the needle cap 136, comprises a chamber 218 that fits over the needle 50. A tortuous path engagement 220 inhibits bacterial or other contamination of the needle 50. At a proximal end 224 of the chamber 218, a flange 222 with a circumferential lip 226 also supports the elongated needle cap 216. In this embodiment, a user reaches slightly into the cavity 174 of the bottle adapter 48′ to grasp and remove the needle cap 216, but the wall 172 still surrounds the needle 50 even with the needle cap 216 removed. With the lid 188 open and the needle cap 216 removed, finger pressure against the wall 172 along the long axis of the distal end 176 will allow a wide mouthed sample bottle to be inserted into the bottle adapter 48′.

On opposing sides of the central through bore 178 in the base 168 are a pair of brackets 228, 230 (see FIG. 10 and FIG. 12) that connect to the latches 78, 80 of the flow control valve 46. A female socket 232 at the proximal side of a needle mount 179 receives the tube segment 64. A cylindrical connector 234 at the distal side of the through bore 178 both receives the needle 50 and contacts the tortuous path engagement 220 of the elongated needle cap 216.

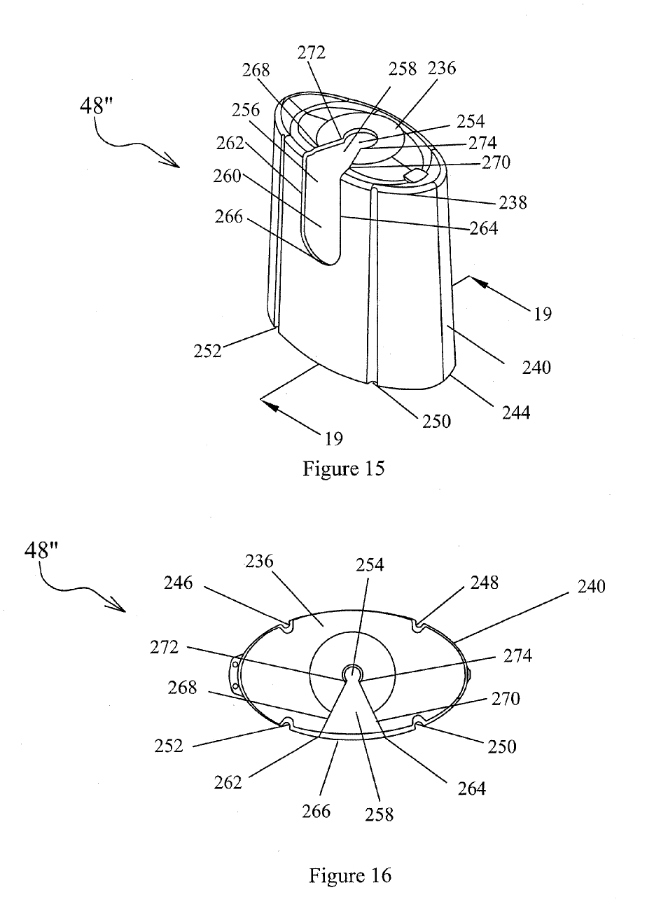

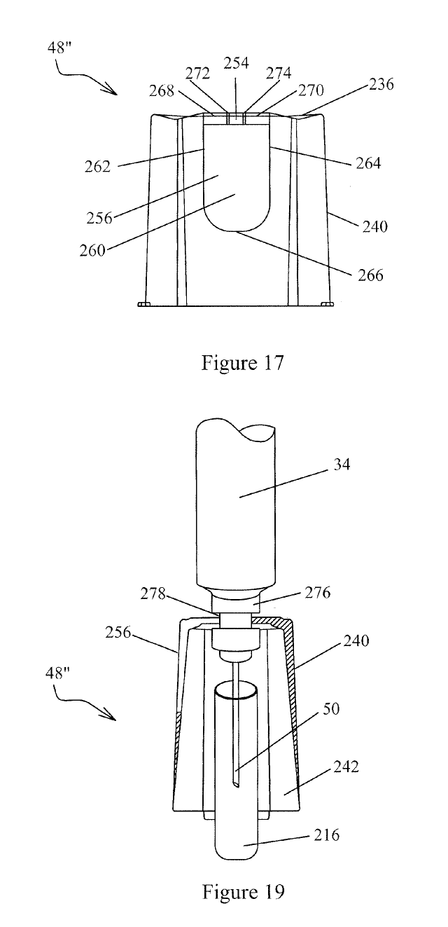

A third embodiment of a bottle adapter 48″ is illustrated in FIGS. 15 through 19. In this embodiment the bottle adapter 48″ can be installed on or removed from the bacterial sampling apparatus 10 without removing a needle cap. The bacterial sampling apparatus 10 can, therefore, be sterilized, packaged and stored without the removable bottle adapter 48″ attached. Shortly before sampling, the removable bottle adapter 48″ can be fitted over the needle 50, which is protected by a needle cap. The cap can then be removed and a sample can be safely taken.

The removable bottle adapter 48″ is shown in perspective view in FIG. 15. In a preferred embodiment, the bottle adapter 48″ also comprises a generally ovoid or elongated base 236 forming a proximal end 238 of the bottle adapter 48″. A wall 240 extends from the base 236, forming a cavity 242. A distal end 244 of the bottle adapter 48″ is open to receive the mouth of a medical sample bottle (not shown). Expandable folds 246, 248, 250, 252 in the wall 240 allow the wall to expand into a round or cylindrical configuration near the distal end 244 of the bottle adapter 48″. Since the bottle adapter 48″ is removable, however, an ovoid or elongated base is not necessary for the assembled sampling system to be inserted in a storage device. The base, therefore, can be circular or any other suitable configuration, and a non-deformable wall may also be used. Moreover, a particular shape for the removable bottle adapter 48″ may be selected to conform to available sample bottles. A set of relatively inexpensive bottle adapters 48″ may be provided with a single sampling apparatus 10 so that the apparatus may be adapted to an available sampling bottle at the point of use.

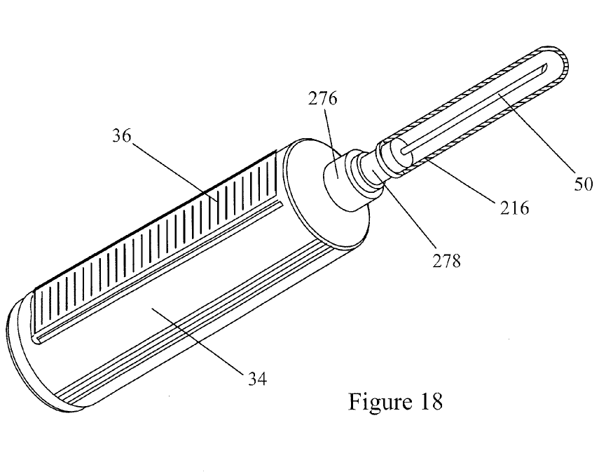

A central though bore 254 in the base 236 provides access for the needle 50 and needle cap (FIG. 18 and FIG. 19). Brackets (not shown in this embodiment), such as brackets 108 and 110, may be placed adjacent the through bore 254, particularly if the bottle adapter 48″ is used with a flow control valve, such as the flow control valve 46 described above and shown, for example, in FIG. 2. The central bore 254 of the removable bottle adapter 48″ opens into a slot 256 that comprises a triangular opening 258 in the base 236 and a longitudinally extending, generally rectangular opening 260 in the wall 240. The longitudinal opening 260 has two parallel edges 262, 264 connected by a bottom edge 266. The bottom edge 266 may be curved, as shown, or linear. The triangular opening 260 comprises two converging edges 268, 270 that extend from the parallel edges 262, 264, respectively, towards the central bore 254. The converging edges 268, 270 intersect the bore 254 such that two opposed prominences 272, 274 are formed. When a needle and needle holder are inserted into the bottle adapter 48″, the prominences 272, 274 hold the needle in place in the central bore 254 of the bottle adapter 48″.

In connection with this third embodiment, the reservoir 34, needle 50 and needle holder 256 may be used without a flow control valve 46, as shown in FIG. 18 and FIG. 19, or with a flow control valve 46, as described above. The reservoir 34 may be connected to the needle 50 through a coupling 276 having a neck 278 of reduced diameter. As shown in FIG. 19 the reduced diameter neck 278 allows the coupling 276 to snap into the central bore 254 and to be held in place by the prominences 272, 274. With the needle cap 256 mounted on the connector 276 as shown in FIG. 18, the needle cap 256 and needle 50 can be inserted into the bottle adapter 48″ through the slot 256 and snapped into place. The needle cap 256 can then be removed, as shown in FIG. 19, exposing the needle 50, which is still protected by the bottle adapter 48″.

It should be understood that various changes and modifications to the presently preferred embodiments described herein will be apparent to those skilled in the art. It is foreseeable that the shape of the reservoir, as well as the configuration of the flow control clamp may be varied. It is also foreseeable that the apparatus may be manufactured out of a plurality of different polymeric materials. These examples are not meant to be limiting, but rather are exemplary of the modifications that can be made without departing from the spirit and scope of the present invention and without diminishing its attendant advantages.

What is claimed is:

1. A biologic fluid sampling device comprising
a biologically compatible reservoir having an interior chamber for receiving a biologic fluid, a fluid access port, and a fluid egress port,
a needle in fluid communication with said chamber through said fluid egress port, and
a valve interposed between said chamber and said needle whereby flow of biologic fluid from said chamber through said needle may be selectively controlled.

2. The sampling device according to claim 1 further comprising a rigid base supporting said valve, said base being coupled to said reservoir and to said needle and supporting said needle in fixed relationship with respect to said reservoir.

3. The sampling device according to claim 2 wherein said rigid base has at least one side wall coupled to said base.

4. The sampling device according to claim 3 wherein said rigid base has a central beam extending generally linearly from said fluid egress port to said needle.

5. The sampling device a according to claim 4 further comprising a flexible tube extending along said central beam from said fluid egress port to said needle and wherein said valve comprises an arm pivotally connected to said rigid connector and extending across tube and said central beam such that said tube can be compressed between said beam and said arm.

6. The sampling device according to claim 5 wherein said arm has a fixed end and a movable end and said device further comprises a bracket mounted on said connector, said moveable end of said arm being connected to said bracket, and a hinge mounted on said connector, said fixed end of said arm being connected to said hinge.

7. The sampling device according to claim 6 wherein said bracket further comprises a pair of opposed latches and said moveable end of said arm further comprises a tab configured to be captured between said latches.

8. The sampling device according to claim 7 further comprising a ridge extending along said arm, said ridge being configured to selectively press against said tube.

9. The sampling device according to claim 1 further comprising a bottle adapter connected to said valve and surrounding said needle.

10. The sampling device according to claim 9 wherein said bottle adapter further comprises a needle mount for connecting said needle, an elongated base circumferentially surrounding said needle mount and attached thereto, a wall connected to said base and extending along said needle such that said needle is enclosed within said bottle adapter, and a plurality of inwardly extending, longitudinal fins connected to said wall and spaced around said needle mount.

11. The sample device according to claim 10 wherein said fins form a generally cylindrical space centered on said needle.

12. The sampling device according to claim 11 wherein said bottle adaptor further comprises an elliptical lid pivotally connected to said wall distal from said base at a hinge.

13. The sampling device according to claim 12 further comprising a needle cap removably fitted over said needle within said bottle adaptor.

14. The sampling device according to claim 13 wherein said needle cap further comprises a grip extending from a distal end of said needle cap, said grip configured to extend through a slot in said lid when said lid is closed on said bottle adapter.

15. The sampling device according to claim 9 wherein said bottle adapter further comprises a needle mount for connecting said needle, a base circumferentially surrounding said needle mount and attached thereto, a wall connected to said base and extending along said needle such that said needle is enclosed within said bottle adaptor, said wall being deformable from a first configuration into a second configuration.

16. The sampling device according to claim 15 wherein in said first configuration of said bottle adaptor, said wall is generally elliptical and in said second configuration said wall is generally circular.

17. The sampling device according to claim 16 wherein said wall of said bottle adaptor further comprises at least one longitudinally extending expandable fold.

18. The sampling device according to claim 17 wherein said wall of said bottle adaptor further comprises a plurality of expandable folds, said folds being symmetrically spaced around said wall.

19. The sampling device according to claim 9 wherein said bottle adaptor further comprises an elliptical lid pivotally connected to said wall distal from said base at a hinge.

20. The sampling device according to claim 19 wherein said bottle adaptor further comprises a latch mounted on said wall adjacent said hinge and said lid further comprises a catch adapted to engage said latch, whereby said lid is secured in an open position along said wall.

21. The sampling device according to claim 9 further comprising a needle cap removably fitted over said needle within said bottle adaptor.

22. The sampling device according to claim 21 wherein said bottle adaptor further comprises a needle mount for connecting said needle, a base circumferentially surrounding said needle mount and attached thereto, a wall connected to said base and extending along said needle such that said needle is enclosed within said bottle adaptor, said needle mount comprising a cylindrical connector, said needle cap being adapted to connect to said cylindrical connector.

23. The sampling device according to claim 22 wherein said bottle adaptor further comprises a lid pivotally connected to said wall by a hinge and a clasp connected to said wall for securing said lid in a closed position, said lid having a centrally located protrusion, said protrusion contacting a distal end of said needle cap when said lid is in said closed position, said protrusion forcing said cap onto said male connector.

24. The sampling device according to claim 21 wherein said needle cap further comprises a grip extending from a distal end of said needle cap, said grip configured to extend through a slot in said lid when said lid is closed on said bottle adapter.

25. The sampling device according to claim 1 further comprising a biologic fluid product bag, a tube connecting said bag to said fluid access port of said biologically compatible receptacle, and a frangible valve interposed in said tube.

26. The sampling device according to claim 1 further comprising a coupling at a proximal end of said needle, a needle cap removably fitted over said needle and releasably connected to said coupling, and a bottle adaptor configured to removably connect to said coupling.

27. The sampling device of claim 26, wherein said bottle adaptor comprises a centrally located bore, said coupling being adapted to fit within said bore, and a slot extending lengthwise from said bore such that said needle and said needle cap can be inserted into said bottle adaptor through said slot.

28. The sampling device of claim 27, wherein said coupling comprises a neck.

29. A biologic fluid sampling device comprising
a biologically compatible receptacle having an interior chamber for receiving a biologic fluid, a fluid access port, and a fluid egress port,
a needle in fluid communication with said chamber through said fluid egress port, and
a bottle adapter connected to said valve and surrounding said needle, said bottle adapter having a needle mount for connecting said needle, a base circumferentially surrounding said needle mount and attached thereto, a wall connected to said base and extending along said needle such that said needle is enclosed within said bottle adaptor, and a plurality of inwardly extending, longitudinal fins connected to said wall and spaced around said needle mount.

30. The sample device according to claim 29 wherein said fins form a generally cylindrical space centered on said needle.

31. The sampling device according to claim 30 wherein said bottle adaptor further comprises an elliptical lid pivotally connected to said wall distal from said base at a hinge.

32. The sampling device according to claim 31 further comprising a needle cap removably fitted over said needle within said bottle adaptor.

33. The sampling device according to claim 32 wherein said needle cap further comprises a grip extending from a distal end of said needle cap, said grip configured to extend through a slot in said lid when said lid is closed on said bottle adapter.

34. A biologic fluid sampling device comprising
a biologically compatible receptacle having an interior chamber for receiving a biologic fluid, a fluid access port, and a fluid egress port,
a needle in fluid communication with said chamber through said fluid egress port, and
a bottle adapter connected to said valve and surrounding said needle, said bottle adapter having a needle mount for connecting said needle, a base circumferentially surrounding said needle mount and attached thereto, a wall connected to said base and extending along said needle such that said needle is enclosed within said bottle adaptor, said wall being deformable from a first configuration into a second configuration.

35. The sampling device according to claim 34 wherein in said first configuration of said bottle adaptor, said wall is generally elliptical and in said second configuration said wall is generally circular.

36. The sampling device according to claim 35 wherein said wall of said bottle adaptor further comprises at least one longitudinally extending, expandable fold.

37. The sampling device according to claim 36 wherein said wall of said bottle adaptor further comprises a plurality of expandable folds, said folds being symmetrically spaced around said wall.

38. The sampling device according to claim 37 wherein said bottle adaptor further comprises an elliptical lid pivotally connected to said wall distal from said base at a hinge.

39. The sampling device according to claim 38 wherein said bottle adaptor further comprises a latch mounted on said wall adjacent said hinge and said lid further comprises a catch adapted to engage said latch, whereby said lid is secured in an open position along said wall.

40. The sampling device according to claim 34 further comprising a needle cap removably fitted over said needle within said bottle adaptor.

41. The sample device according to claim 40 wherein said bottle adaptor further comprises a needle mount for connecting said needle, a base circumferentially surrounding said needle mount and attached thereto, a wall connected to said base and extending along said needle such that said needle is enclosed within said bottle adaptor, said needle mount comprising a cylindrical connector, said needle cap being adapted to connect to said cylindrical connector.

42. The sample device according to claim 41 wherein said bottle adaptor further comprises a lid pivotally connected to said wall by a hinge and a clasp connected to said wall for securing said lid in a closed position, said lid having a centrally located protrusion, said protrusion contacting a distal end of said needle cap when said lid is in said closed position, said protrusion forcing said cap onto said cylindrical connector.

43. A biologic fluid sampling device comprising
a biologically compatible receptacle having an interior chamber for receiving a biologic fluid, a fluid access port, and a fluid egress port,
a needle in fluid communication with said chamber through said fluid egress port, and
a bottle adapter connected to said valve and surrounding said needle, said bottle adapter having a needle mount for connecting said needle, a base circumferentially surrounding said needle mount and attached thereto, a wall connected to said base and extending along said needle such that said needle is enclosed within said bottle adaptor, and
a needle cap removably fitted over said needle within said bottle adaptor.

44. The sampling device according to claim 43 wherein said bottle adaptor further comprises a needle mount for connecting said needle, a base circumferentially surrounding said needle mount and attached thereto, a wall connected to said base and extending along said needle such that said needle is enclosed within said bottle adaptor, a cylindrical connector mounted on said base and circumferentially surrounding said needle mount, said needle cap being adapted to connect to said cylindrical connector.

45. The sample device according to claim 44 wherein said bottle adaptor further comprises a lid pivotally connected to said wall by a hinge and a clasp connected to said wall for securing said lid in a closed position, said lid having a centrally located protrusion, said protrusion contacting a distal end of said needle cap when said lid is in said closed position, said protrusion forcing said cap onto said connector.

46. A biologic fluid sampling device comprising
a biologically compatible receptacle having an interior chamber for receiving a biologic fluid, a fluid access port, and a fluid egress port,
a needle in fluid communication with said chamber through said fluid egress port,
a collar at a proximal end of said needle,
a needle cap removably fitted over said needle and releasably connected to said collar, and
a bottle adaptor configured to removably connect to said collar.

47. The sampling device of claim 46, wherein said bottle adaptor comprises a centrally located bore, said collar being adapted to fit within said bore, and a slot extending lengthwise from said bore such that said needle and said needle cap can be inserted into said bottle adaptor through said slot.

48. The sampling device of claim 47, wherein said coupling comprises a neck.
